(19) United States
(12) Patent Application Publication
(10) Pub. No.: US 2020/0297711 A1
(43) Pub. Date: Sep. 24, 2020

(54) PHARMACEUTICAL COMPOSITION COMPRISING FGFR SELECTIVE TYROSINE KINASE INHIBITOR

(71) Applicant: Eisai R&D Management Co., Ltd., Bunkyo-ku, Tokyo (JP)

(72) Inventors: Tatsuya Sasaki, Tokyo (JP); Kenichi Saito, Woodcliff Lake, NJ (US)

(21) Appl. No.: 16/642,105

(22) PCT Filed: Oct. 10, 2018
(86) PCT No.: PCT/JP2018/037690

§ 371 Date: Feb. 26, 2020

Related U.S. Application Data
(60) Provisional application No. 62/571,391, filed on Oct. 12, 2017.

(51) Int. Cl. A61K 31/4545; A61K 9/00; A61P 35/04

(52) U.S. Cl. CPC A61K 31/4545; A61P 35/04; A61K 9/0053

(57) ABSTRACT

The present invention provides a pharmaceutical composition comprising FGFR selective tyrosine kinase inhibitor, specifically 5-((2-(4-(1-(2-hydroxyethyl)piperidin-4-yl)benzamide)pyridine-4-yl)oxy)-6-(2-methoxyethoxy)-N-methyl-1H-indole-1-carboxamide or a pharmaceutically acceptable salt thereof.

### TECHNICAL FIELD

The present invention relates to a pharmaceutical composition comprising FGFR selective tyrosine kinase inhibitor, specifically 5-((2-(4-(1-(2-hydroxyethyl)piperidin-4-yl)benzamide)pyridine-4-yl)oxy)-6-(2-methoxyethoxy)-N-methyl-1H-indole-1-carboxamide or a pharmaceutically acceptable salt thereof.

### BACKGROUND ART

The compound represented by the formula (I):

### [Chem. 1]

[symbol]

is known as 5-((2-(4-(1-(2-hydroxyethyl)piperidin-4-yl)benzamide)pyridin-4-yl)oxy)-6-(2-methoxyethoxy)-N-methyl-1H-indole-1-carboxamide (hereinafter referred to as “Compound A”). It has been reported that Compound A has an inhibitory activity on fibroblast growth factor receptors (FGFRs) 1, 2 and 3 and has a cell growth suppressing activity in stomach cancer, lung cancer, bladder cancer and endometrial cancer (PTL 1) as well as in bile duct cancer (PTL 2) and in breast cancer (PTL 3).

### CITATION LIST

### Patent Literature

[PTL 1] US 2014-235614

[PTL 2] WO/2016/152907

[PTL 3] WO/2017/104739

### SUMMARY OF INVENTION

### Technical Problem

The relationship between the pharmacokinetics (hereinafter referred to as “PK”) of Compound A in human subjects and the therapeutically effective amount thereof to be expected has not been known.

In addition, although the structure of 6-(2-Methoxyethoxy)-N-methyl-5-((2-(4-(piperidin-4-yl)benzamide)pyridin-4-yl)oxy)-1H-indole-1-carboxamide (hereinafter referred to as “Compound B”) and the FGFR inhibitory activity thereof in preclinical model have been disclosed in PTL 1, it has not been known that when Compound A is administered to human subjects, Compound A is metabolized in the body to produce Compound B. The compound B is represented by the formula (II):

### [Chem. 2]

[symbol]

### Solution to Problem

It is an object of the present invention to provide a pharmaceutical composition comprising a therapeutically effective amount of Compound A or pharmaceutically acceptable salts thereof.

The present invention relates to the following 21 1> to <22>.

<1> An oral dosage form comprising about 30 mg to about 140 mg of Compound A or a pharmaceutically acceptable salt thereof and at least one pharmaceutically acceptable excipient, wherein said Compound A is 5-((2-(4-(1-(2-hydroxyethyl)piperidin-4-yl)benzamide)pyridine-4-yl)oxy)-6-(2-methoxyethoxy)-N-methyl-1H-indole-1-carboxamide represented by Formula (I):

### [Chem. 3]

[symbol]

<2> The oral dosage form of <1>, wherein said oral dosage form at a single daily dose achieves a mean Cₘₐₓ of said Compound A of from about 28 ng/mL to about 3.5×10² ng/mL after administration to human subjects.

<3> The oral dosage form of <1>, wherein said oral dosage form at a single daily dose achieves a mean AUC₍₀₋ₜ₎ of said Compound A of from about 2.2×10² h*ng/mL to about 7.2×10³ h*ng/mL after administration to human subjects.

<4> The oral dosage form of <1>, wherein said oral dosage form at a single daily dose achieves a mean AUC_{(0-inf)} of said Compound A of from about 2.3×10² h*ng/mL to about 7.4×10³ h*ng/mL after administration to human subjects.

<5> The oral dosage form of <1>, wherein said oral dosage form at a single daily dose achieves a mean Cₘₐₓ of Compound B of from about 19 ng/mL to about 1.0×10² ng/mL after administration to human subjects, and said Compound B is 6-(2-Methoxyethoxy)-N-methyl-5-((2-(4-(piperidin-4-yl)benzamide)pyridin-4-yl)oxy)-1H-indole-1-carboxamide represented by Formula (II):

### [Chem. 4]

[symbol]

<6> The oral dosage form of <1>, wherein said oral dosage form at a single daily dose achieves a mean AUC₍₀₋ₜ₎ of Compound B of from about 2.7x10² h*ng/mL to about 1.6×10³ h*ng/mL after administration to human subjects, and said Compound B is 6-(2-Methoxyethoxy)-N-methyl-5-((2-(4-(piperidin-4-yl)benzamide)pyridin-4-yl)oxy)-1H-indole-1-carboxamide represented by Formula (II):

### [Chem. 5]

[symbol]

<7> The oral dosage form of <1>, wherein said oral dosage form at a single daily dose achieves a mean AUC_{(0-inf)} of Compound B of from about 2.9×10² h*ng/mL to about 1.7×10³ h*ng/mL after administration to human subjects, and said Compound B is 6-(2-Methoxyethoxy)-N-methyl-5-((2-(4-(piperidin-4-yl)benzamide)pyridin-4-yl)oxy)-1H-indole-1-carboxamide represented by Formula (II):

### [Chem. 6]

[symbol]

<8> An oral dosage form comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said Compound A is 5-((2-(4-(1-(2-hydroxyethyl)piperidin-4-yl)benzamide)pyridine-4-yl)oxy)-6-(2-methoxyethoxy)-N-methyl-1H-indole-1-carboxamide represented by Formula (I):

### [Chem. 7]

[symbol]

<9> The oral dosage form of <8>, wherein said single daily dose achieves a mean Cₘₐₓ of said Compound A of from about 28 ng/mL to about 3.5×10² ng/mL after administration to human subjects.

<10> The oral dosage form of <8>, wherein said single daily dose achieves a mean AUC₍₀₋ₜ₎ of said Compound A of from about 2.2×10² h*ng/mL to about 7.2×10³ h*ng/mL after administration to human subjects.

<11> The oral dosage form of <8>, wherein said single daily dose achieves a mean AUC_{(0-inf)} of said Compound A of from about 2.3×10² h*ng/mL to about 7.4×10³ h*ng/mL after administration to human subjects.

<12> The oral dosage form of <8>, wherein said single daily dose achieves a mean Cₘₐₓ of Compound B of from about 19 ng/mL to about 1.0×10² ng/mL after administration to human subjects, and said Compound B is 6-(2-Methoxyethoxy)-N-methyl-5-((2-(4-(piperidin-4-yl)benzamide)pyridin-4-yl)oxy)-1H-indole-1-carboxamide represented by Formula (II):

### [Chem. 8]

[symbol]

<13> The oral dosage form of <8>, wherein said single daily dose achieves a mean AUC₍₀₋ₜ₎ of Compound B of from about 2.7×10² h*ng/mL to about 1.6×10³ h*ng/mL after administration to human subjects, and said Compound B is 6-(2-Methoxyethoxy)-N-methyl-5-((2-(4-(piperidin-4-yl)benzamide)pyridin-4-yl)oxy)-1H-indole-1-carboxamide represented by Formula (II):

### [Chem. 9]

[symbol]

<14> The oral dosage form of <8>, wherein said single daily dose achieves a mean AUC_{(0-inf)} of Compound B of from about 2.9×10² h*ng/mL to about 1.7×10³ h*ng/mL after administration to human subjects, and said Compound B is 6-(2-Methoxyethoxy)-N-methyl-5-((2-(4-(piperidin-4-yl)benzamide)pyridin-4-yl)oxy)-1H-indole-1-carboxamide represented by Formula (II):

### [Chem. 10]

[symbol]

<15> The oral dosage form of <1> or <14>, wherein said oral dosage form is used for treatment of stomach cancer, lung cancer, bladder cancer, endometrial cancer, bile duct cancer or breast cancer.

<16> A method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said Compound A is 5-((2-(4-(1-(2-hydroxyethyl)piperidin-4-yl)benzamide)pyridine-4-yl)oxy)-6-(2-methoxyethoxy)-N-methyl-1H-indole-1-carboxamide represented by Formula (I):

### [Chem. 11]

[symbol]

<17> The method of <16>, wherein said single daily dose achieves Cₘₐₓ of said Compound A of from about 28 ng/mL to about 3.5×10² ng/mL after administration to the human subject.

<18> The method of <16>, wherein said single daily dose achieves AUC₍₀₋ₜ₎ of said Compound A of from about 2.2×10² h*ng/mL to about 7.2×10³ h*ng/mL after administration to the human subject.

<19> The method of <16>, wherein said single daily dose achieves AUC_{(0-inf)} of said Compound A of from about 2.3×10² h*ng/mL to about 7.4×10³ h*ng/mL after administration to the human subject.

<20> The method of <16>, wherein said single daily dose achieves a mean Cₘₐₓ of Compound B of from about 19 ng/mL to about 1.0×10² ng/mL after administration to human subjects, and said Compound B is 6-(2-Methoxyethoxy)-N-methyl-5-((2-(4-(piperidin-4-yl)benzamide)pyridin-4-yl)oxy)-1H-indole-1-carboxamide represented by Formula (II):

### [Chem. 12]

[symbol]

<21> The method of <16>, wherein said single daily dose achieves a mean AUC₍₀₋ₜ₎ of Compound B of from about 2.7×10² h*ng/mL to about 1.6×10³ h*ng/mL after administration to human subjects and said Compound B is 6-(2-Methoxyethoxy)-N-methyl-5-((2-(4-(piperidin-4-yl)benzamide)pyridin-4-yl)oxy)-1H-indole-1-carboxamide represented by Formula (II):

### [Chem. 13]

[symbol]

<22> The method of <16>, wherein said single daily dose achieves a mean AUC_{(0-inf)} of said Compound B of from about 2.9×10² h*ng/mL to about 1.7×10³ h*ng/mL after administration to human subjects and said Compound B is 6-(2-Methoxyethoxy)-N-methyl-5-((2-(4-(piperidin-4-yl)benzamide)pyridin-4-yl)oxy)-1H-indole-1-carboxamide represented by Formula (II):

### [Chem. 14]

[symbol]

### BRIEF DESCRIPTION OF DRAWINGS

FIG. 1 shows plasma concentration profiles of Compound A following a single dose of Compound A.

FIG. 2 shows plasma concentration profiles of Compound A following repeated doses of Compound A.

FIG. 3 shows CT images of the patient with FGFR2 gene amplified diffused type gastric cancer before the administration of Compound A (left) and on Day 1 of Cycle 3 (right).

FIG. 4 shows percent change from baseline on Day 15 of Cycle 1 of PD markers.

Left: phosphate, middle: FGF23, and right: 1,25-dihydroxyvitamin D₃.

### DESCRIPTION OF EMBODIMENTS

### I. Definitions

In order the invention described herein may be more fully understood, the following definitions are provided for the purposes of the disclosure:

The term “effective amount” means an amount of Compound A that is capable of achieving a therapeutic effect in a human subjective in need thereof.

The term “human subject” shall mean a normal healthy male or female volunteers and/or any individual that presents with clinical signs or symptoms of cancer.

The expression “bioequivalent” or “bioequivalence” is a term of art and is intended to be defined in accordance with Approved Drug Products with Therapeutic Equivalence Evaluations, 34th Edition, which is published by the U.S. Department of Health and Human Services, and is commonly known as the “Orange Book”. Bioequivalence of different formulation of the same drug substance involves equivalence with respect to the rate and extent of drug absorption. The extent and rate of absorption of the test formulation is compared to a reference formulation in order to determine whether the two formulations are bioequivalent. The standard bioequivalence study is conducted in crossover fashion by extensive testing which includes administering single doses of the test and reference drugs to a number of volunteers, usually 12 to 24 healthy normal adults, and then measuring the blood or plasma levels of the drug over time. Detailed guidelines for establishing the bioequivalence of a formulation with a reference formulation have been published by the FDA Office of Generic Drugs, Division of Bioequivalence.

Two formulations whose PK parameters such as Cₘₐₓ, AUC, or tₘₐₓ differ by −20%/+25% or less are generally considered to be “bioequivalent”. Another approach for average bioequivalence involves the calculation of a 90% confidence interval for the ratio of the averages (population geometric means) of the measures for the test and reference products. To establish bioequivalence, the calculated confidence interval should fall within usually 80-125% for the ratio of the product averages. In addition to this general approach, the others approach, including (1) logarithmic transformation of pharmacokinetic data, (2) methods to evaluate sequence effects and (3) methods to evaluate outlier data, may be useful for the establishment of bioequivalence. For example, in the above (1) the confidence interval should fall within usually 80-125% for the difference in the mean value of the logarithmic converted PK parameter.

The term “dosage form(s)” shall mean the means to administer the drug substance (active pharmaceutical ingredient (API)), or to facilitate dosing, administration, and delivery of the medicine to the patient and other mammals Dosage forms are classified in terms of administration routes and application sites, including, for example, oral, topical, rectal, vaginal, intravenous, subcutaneous, intramuscular, ophthalmic, nasal, otic and inhalation administration. Alternatively, dosage forms are classified in terms of physical form such as solid, semi-solid or liquid. Furthermore, dosage forms are subdivided based on their form, functions and characteristics, including, without limited, tablet, capsule or injection as described in monograph of Japanese Pharmacopoeia 16 edition (JP16) or General Chapter <1151> Pharmaceutical Dosage Forms of U.S. Pharmacopoeia-NF (37)(USP37).

The term “excipient” shall mean a typically inactive ingredient used as a vehicle (for example, water, capsule shell etc.), a diluent, or a component to constitute a dosage form or pharmaceutical composition comprising a drug such as a therapeutic agent. The term also encompasses a typically inactive ingredient that imparts cohesive function (i.e. binder), disintegrating function (i.e. disintegrator), lubricant function (lubricating agent), and/or the other function (i.e. solvent, surfactant etc.) to the composition.

The term “a mean” refers to an arithmetical mean. The pharmacokinetic parameters such as “a mean Cₘₐₓ” or “a mean AUC” refer to the arithmetical mean value of a Cₘₐₓ or an AUC.

The list of the abbreviations and definitions of the terms used in this application is presented the following.
AUC: Area under the plasma concentration-time curve
AUC₍₀₋ₜ₎: Area under the plasma concentration-time curve from time zero to time of last quantifiable concentration
   AUC_{(0-inf)}: Area under the plasma concentration-time curve from time zero to infinite time
   CL/F: Apparent total clearance following extravascular (e.g., oral) administration
   Cₘₐₓ: Maximum observed concentration
   t_{1/2}: Terminal elimination half-life
   tₘₐₓ: Time to reach maximum (peak) concentration following drug administration
   Vz/F: Apparent volume of distribution at terminal phase

The term “about”, unless otherwise indicated, refers to a value that is no more than 10% above or below the value being modified by the term. For example, the term “about 30 mg” means a range of from 27 mg to 33 mg.

The amount of Compound A or a pharmaceutically acceptable salt thereof contained in the oral dosage form is represented as the amount of Compound A in free form. For example, the term “an oral dosage form comprising about 30 mg of Compound A or a pharmaceutically acceptable salt thereof” means that the oral dosage form comprises Compound A or a pharmaceutically acceptable salt thereof equivalent to about 30 mg of Compound A in free form. When the oral dosage form is in the form of a dosage unit containing a particular amount of Compound A or a pharmaceutically acceptable salt thereof such as tablet and capsule, one or more dosage units may provide the amount of Compound A or a pharmaceutically acceptable salt thereof contained in the oral dosage form. For example, the term “an oral dosage form comprising about 30 mg of Compound A or a pharmaceutically acceptable salt thereof” means that the amount of Compound A or a pharmaceutically acceptable salt thereof contained in one dosage unit may be about 30 mg or that the amount of Compound A or a pharmaceutically acceptable salt thereof in two or more dosage unit may be about 30 mg in total.

### II. Description of the Embodiments

In one embodiment, the present invention provides an oral dosage form comprising about 30 mg to about 140 mg of Compound A or a pharmaceutically acceptable salt thereof and at least one pharmaceutically acceptable excipient.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at a single daily dose achieves a mean Cₘₐₓ of said Compound A of from about 28 ng/mL to about 3.5×10² ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at a single daily dose achieves a mean Cₘₐₓ of said Compound A of from about 28 ng/mL to about 2.3×10² ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at a single daily dose achieves a mean AUC₍₀₋ₜ₎ of said Compound A of from about 2.2×10² h*ng/mL to about 7.2×10³ h*ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at a single daily dose achieves a mean AUC₍₀₋ₜ₎ of said Compound A of from about 2.2×10² h*ng/mL to about 4.0×10³ h*ng/mL after administration to human subjects. In one embodiment, the present invention provides an oral dosage form comprising about 30 mg to about 140 mg of Compound A or a pharmaceutically acceptable salt thereof and at least one pharmaceutically acceptable excipient, wherein said oral dosage form at a single daily dose achieves a mean AUC₍₀₋ₜ₎ of said Compound A of from about 2.2×10² h*ng/mL to about 3.9×10³ h*ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at a single daily dose achieves a mean AUC_{(0-inf)} of said Compound A of from about 2.3×10² h*ng/mL to about 7.4×10³ h*ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at a single daily dose achieves a mean AUC_{(0-inf)} of said Compound A of from about 2.3×10² h*ng/mL to about 4.1×10³ h*ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at a single daily dose achieves a mean AUC_{(0-inf)} of said Compound A of from about 2.3×10² h*ng/mL to about 4.0×10³ h*ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at a single daily dose achieves a mean Cₘₐₓ of said Compound B of from about 19 ng/mL to about 1.0×10² ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at a single daily dose achieves a mean Cₘₐₓ of said Compound B of from about 19 ng/mL to about 64 ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at a single daily dose achieves a mean AUC₍₀₋ₜ₎ of said Compound B of from about 2.7×10² h*ng/mL to about 1.6×10³ h*ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at a single daily dose achieves a mean AUC₍₀₋ₜ₎ of said Compound B of from about 2.7×10² h*ng/mL to about 1.4×10³ h*ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at a single daily dose achieves a mean AUC_{(0-inf)} of said Compound B of from about 2.9×10² h*ng/mL to about 1.7×10³ h*ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at a single daily dose achieves a mean AUC_{(0-inf)} of said Compound B of from about 2.9×10² h*ng/mL to about 1.5×10³ h*ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at repeated once-daily doses achieves a mean Cₘₐₓ of said Compound A of from about 13 ng/mL to about 5.5×10² ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at repeated once-daily doses achieves a mean Cₘₐₓ of said Compound A of from about 13 ng/mL to about 3.8×10² ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at repeated once-daily doses achieves a mean AUC₍₀₋ₜ₎ of said Compound A of from about 1.5×10² h*ng/mL to about 8.1×10³ h*ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at repeated once-daily doses achieves a mean AUC₍₀₋ₜ₎ of said Compound A of from about 1.6×10² h*ng/mL to about 8.1×10³ h*ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at repeated once-daily doses achieves a mean AUC₍₀₋ₜ₎ of said Compound A of from about 1.5×10² h*ng/mL to about 4.7×10³ h*ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at repeated once-daily doses achieves a mean AUC₍₀₋ₜ₎ of said Compound A of from about 1.6×10² h*ng/mL to about 4.7×10³ h*ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at repeated once-daily doses achieves a mean Cₘₐₓ of said Compound B of from about 12 ng/mL to about 1.6×10² ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at repeated once-daily doses achieves a mean Cₘₐₓ of said Compound B of from about 12 ng/mL to about 1.1×10² ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at repeated once-daily doses achieves a mean AUC₍₀₋ₜ₎ of said Compound B of from about 1.9×10² h*ng/mL to about 2.1×10³ h*ng/mL after administration to human subjects.

In one embodiment, the present invention provides an oral dosage form comprising about 30 mg to about 140 mg of Compound A or a pharmaceutically acceptable salt thereof and at least one pharmaceutically acceptable excipient, wherein said oral dosage form at repeated once-daily doses achieves a mean AUC₍₀₋ₜ₎ of said Compound B of from about 1.9×10² h*ng/mL to about 1.7×10³ h*ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at a single daily dose achieves a mean Cₘₐₓ of said Compound A of from about 28 ng/mL to about 3.5×10² ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at a single daily dose achieves a mean Cₘₐₓ of said Compound A of from about 28 ng/mL to about 2.3×10² ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at a single daily dose achieves a mean AUC₍₀₋ₜ₎ of said Compound A of from about 2.2×10² h*ng/mL to about 7.2×10³ h*ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at a single daily dose achieves a mean AUC₍₀₋ₜ₎ of said Compound A of from about 2.2×10² h*ng/mL to about 4.0×10³ h*ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at a single daily dose achieves a mean AUC₍₀₋ₜ₎ of said Compound A of from about 2.2×10² h*ng/mL to about 3.9×10³ h*ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at a single daily dose achieves a mean AUC_{(0-inf)} of said Compound A of from about 2.3×10² h*ng/mL to about 7.4×10³ h*ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at a single daily dose achieves a mean AUC_{(0-inf)} of said Compound A of from about 2.3×10² h*ng/mL to about 4.1×10³ h*ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at a single daily dose achieves a mean AUC_{(0-inf)} of said Compound A of from about 2.3×10² h*ng/mL to about 4.0×10³ h*ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at a single daily dose achieves a mean Cₘₐₓ of said Compound B of from about 19 ng/mL to about 1.0×10² ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at a single daily dose achieves a mean Cₘₐₓ of said Compound B of from about 19 ng/mL to about 64 ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at a single daily dose achieves a mean AUC₍₀₋ₜ₎ of said Compound B of from about 2.7×10² h*ng/mL to about 1.6×10³ h*ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at a single daily dose achieves a mean AUC₍₀₋ₜ₎ of said Compound B of from about 2.7×10² h*ng/mL to about 1.4×10³ h*ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at a single daily dose achieves a mean AUC_{(0-inf)} of said Compound B of from about 2.9×10² h*ng/mL to about 1.7×10³ h*ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at a single daily dose achieves a mean AUC_{(0-inf)} of said Compound B of from about 2.9×10² h*ng/mL to about 1.5×10³ h*ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at repeated once-daily doses achieves a mean Cₘₐₓ of said Compound A of from about 13 ng/mL to about 5.5×10² ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at repeated once-daily doses achieves a mean Cₘₐₓ of said Compound A of from about 13 ng/mL to about 3.8×10² ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at repeated once-daily doses achieves a mean AUC₍₀₋ₜ₎ of said Compound A of from about 1.5×10² h*ng/mL to about 8.1×10³ h*ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at repeated once-daily doses achieves a mean AUC₍₀₋ₜ₎ of said Compound A of from about 1.6×10² h*ng/mL to about 8.1×10³ h*ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at repeated once-daily doses achieves a mean AUC₍₀₋ₜ₎ of said Compound A of from about 1.5×10² h*ng/mL to about 4.7×10³ h*ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at repeated once-daily doses achieves a mean AUC₍₀₋ₜ₎ of said Compound A of from about 1.6×10² h*ng/mL to about 4.7×10³ h*ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at repeated once-daily doses achieves a mean Cₘₐₓ of said Compound B of from about 12 ng/mL to about 1.6×10² ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at repeated once-daily doses achieves a mean Cₘₐₓ of said Compound B of from about 12 ng/mL to about 1.1×10² ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at repeated once-daily doses achieves a mean AUC₍₀₋ₜ₎ of said Compound B of from about 1.9×10² h*ng/mL to about 2.1×10³ h*ng/mL after administration to human subjects.

In further embodiment, the present invention provides an oral dosage form for treating stomach cancer, lung cancer, bladder cancer, endometrial cancer, bile duct cancer or breast cancer, comprising about 30 mg to about 140 mg of Compound A or a pharmaceutically acceptable salt thereof and at least one pharmaceutically acceptable excipient, wherein said oral dosage form at repeated once-daily doses achieves a mean AUC₍₀₋ₜ₎ of said Compound B of from about 1.9×10² h*ng/mL to about 1.7×10³ h*ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is single daily dose of about 30 mg to 140 mg.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is single daily dose of about 30 mg to 140 mg, and said single daily dose achieves a mean Cₘₐₓ of said Compound A of from about 28 ng/mL to about 3.5×10² ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is single daily dose of about 30 mg to 140 mg, and said single daily dose achieves a mean Cₘₐₓ of said Compound A of from about 28 ng/mL to about 2.3×10² ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is single daily dose of about 30 mg to 140 mg, and said single daily dose achieves a mean AUC₍₀₋ₜ₎ of said Compound A of from about 2.2×10² h*ng/mL to about 7.2×10³ h*ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is single daily dose of about 30 mg to 140 mg, and said single daily dose achieves a mean AUC₍₀₋ₜ₎ of said Compound A of from about 2.2×10² h*ng/mL to about 4.0×10³ h*ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is single daily dose of about 30 mg to 140 mg, and said single daily dose achieves a mean AUC₍₀₋ₜ₎ of said Compound A of from about 2.2×10² h*ng/mL to about 3.9×10³ h*ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is single daily dose of about 30 mg to 140 mg, and said single daily dose achieves a mean AUC_{(0-inf)} of said Compound A of from about 2.3×10² h*ng/mL to about 7.4×10³ h*ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is single daily dose of about 30 mg to 140 mg, and said single daily dose achieves a mean AUC_{(0-inf)} of said Compound A of from about 2.3×10² h*ng/mL to about 4.1×10³ h*ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is single daily dose of about 30 mg to 140 mg, and said single daily dose achieves a mean AUC_{(0-inf)} of said Compound A of from about 2.3×10² h*ng/mL to about 4.0×10³ h*ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is single daily dose of about 30 mg to 140 mg, and said single daily dose achieves a mean Cₘₐₓ of said Compound B of from about 19 ng/mL to about 1.0×10² ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is single daily dose of about 30 mg to 140 mg, and said single daily dose achieves a mean Cₘₐₓ of said Compound B of from about 19 ng/mL to about 64 ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is single daily dose of about 30 mg to 140 mg, and said single daily dose achieves a mean AUC₍₀₋ₜ₎ of said Compound B of from about 2.7×10² h*ng/mL to about 1.6×10³ h*ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is single daily dose of about 30 mg to 140 mg, and said single daily dose achieves a mean AUC₍₀₋ₜ₎ of said Compound B of from about 2.7×10² h*ng/mL to about 1.4×10³ h*ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is single daily dose of about 30 mg to 140 mg, and said single daily dose achieves a mean AUC_{(0-inf)} of said Compound B of from about 2.9×10² h*ng/mL to about 1.7×10³ h*ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is single daily dose of about 30 mg to 140 mg, and said single daily dose achieves a mean AUC_{(0-inf)} of said Compound B of from about 2.9×10² h*ng/mL to about 1.5×10³ h*ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean Cₘₐₓ of said Compound A of from about 13 ng/mL to about 5.5×10² ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean Cₘₐₓ of said Compound A of from about 13 ng/mL to about 3.8×10² ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean AUC₍₀₋ₜ₎ of said Compound A of from about 1.5×10² h*ng/mL to about 8.1×10³ h*ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean AUC₍₀₋ₜ₎ of said Compound A of from about 1.6×10² h*ng/mL to about 8.1×10³ h*ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean AUC₍₀₋ₜ₎ of said Compound A of from about 1.5×10² h*ng/mL to about 4.7×10³ h*ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean AUC₍₀₋ₜ₎ of said Compound A of from about 1.6×10² h*ng/mL to about 4.7×10³ h*ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean Cₘₐₓ of said Compound B of from about 12 ng/mL to about 1.6×10² ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean Cₘₐₓ of said Compound B of from about 12 ng/mL to about 1.1×10² ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean AUC₍₀₋ₜ₎ of said Compound B of from about 1.9×10² h*ng/mL to about 2.1×10³ h*ng/mL after administration to human subjects.

In another embodiment, the present invention provides an oral dosage form comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean AUC₍₀₋ₜ₎ of said Compound B of from about 1.9×10² h*ng/mL to about 1.7×10³ h*ng/mL after administration to human subjects.

In further embodiment, the present invention provides an oral dosage form for treating stomach cancer, lung cancer, bladder cancer, endometrial cancer, bile duct cancer or breast cancer, comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves a mean Cₘₐₓ of said Compound A of from about 28 ng/mL to about 3.5×10² ng/mL after administration to human subjects.

In further embodiment, the present invention provides an oral dosage form for treating stomach cancer, lung cancer, bladder cancer, endometrial cancer, bile duct cancer or breast cancer, comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves a mean Cₘₐₓ of said Compound A of from about 28 ng/mL to about 2.3×10² ng/mL after administration to human subjects.

In further embodiment, the present invention provides an oral dosage form for treating stomach cancer, lung cancer, bladder cancer, endometrial cancer, bile duct cancer, or breast cancer, comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves a mean AUC₍₀₋ₜ₎ of said Compound A of from about 2.2×10² h*ng/mL to about 7.2×10³ h*ng/mL after administration to human subjects.

In further embodiment, the present invention provides an oral dosage form for treating stomach cancer, lung cancer, bladder cancer, endometrial cancer, bile duct cancer, or breast cancer, comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves a mean AUC₍₀₋ₜ₎ of said Compound A of from about 2.2×10² h*ng/mL to about 4.0×10³ h*ng/mL after administration to human subjects.

In further embodiment, the present invention provides an oral dosage form for treating stomach cancer, lung cancer, bladder cancer, endometrial cancer, bile duct cancer, or breast cancer, comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves a mean AUC₍₀₋ₜ₎ of said Compound A of from about 2.2×10² h*ng/mL to about 3.9×10³ h*ng/mL after administration to human subjects.

In further embodiment, the present invention provides an oral dosage form for treating stomach cancer, lung cancer, bladder cancer, endometrial cancer, bile duct cancer or breast cancer, comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves a mean AUC_{(0-inf)} of said Compound A of from about 2.3×10² h*ng/mL to about 7.4×10³ h*ng/mL after administration to human subjects.

In further embodiment, the present invention provides an oral dosage form for treating stomach cancer, lung cancer, bladder cancer, endometrial cancer, bile duct cancer or breast cancer, comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves a mean AUC_{(0-inf)} of said Compound A of from about 2.3×10² h*ng/mL to about 4.1×10³ h*ng/mL after administration to human subjects.

In further embodiment, the present invention provides an oral dosage form for treating stomach cancer, lung cancer, bladder cancer, endometrial cancer, bile duct cancer or breast cancer, comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves a mean AUC_{(0-inf)} of said Compound A of from about 2.3×10² h*ng/mL to about 4.0×10³ h*ng/mL after administration to human subjects.

In further embodiment, the present invention provides an oral dosage form for treating stomach cancer, lung cancer, bladder cancer, endometrial cancer, bile duct cancer or breast cancer, comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves a mean Cₘₐₓ of said Compound B of from about 19 ng/mL to about 1.0×10² ng/mL after administration to human subjects.

In further embodiment, the present invention provides an oral dosage form for treating stomach cancer, lung cancer, bladder cancer, endometrial cancer, bile duct cancer or breast cancer, comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves a mean Cₘₐₓ of said Compound B of from about 19 ng/mL to about 64 ng/mL after administration to human subjects.

In further embodiment, the present invention provides an oral dosage form for treating stomach cancer, lung cancer, bladder cancer, endometrial cancer, bile duct cancer, or breast cancer, comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves a mean AUC₍₀₋ₜ₎ of said Compound B of from about 2.7×10² h*ng/mL to about 1.6×10³ h*ng/mL after administration to human subjects.

In further embodiment, the present invention provides an oral dosage form for treating stomach cancer, lung cancer, bladder cancer, endometrial cancer, bile duct cancer, or breast cancer, comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves a mean AUC₍₀₋ₜ₎ of said Compound B of from about 2.7×10² h*ng/mL to about 1.4×10³ h*ng/mL after administration to human subjects.

In further embodiment, the present invention provides an oral dosage form for treating stomach cancer, lung cancer, bladder cancer, endometrial cancer, bile duct cancer or breast cancer, comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves a mean AUC_{(0-inf)} of said Compound B of from about 2.9×10² h*ng/mL to about 1.7×10³ h*ng/mL after administration to human subjects.

In further embodiment, the present invention provides an oral dosage form for treating stomach cancer, lung cancer, bladder cancer, endometrial cancer, bile duct cancer or breast cancer, comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves a mean AUC_{(0-inf)} of said Compound B of from about 2.9×10² h*ng/mL to about 1.5×10³ h*ng/mL after administration to human subjects.

In further embodiment, the present invention provides an oral dosage form for treating stomach cancer, lung cancer, bladder cancer, endometrial cancer, bile duct cancer or breast cancer, comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean Cₘₐₓ of said Compound A of from about 13 ng/mL to about 5.5×10² ng/mL after administration to human subjects.

In further embodiment, the present invention provides an oral dosage form for treating stomach cancer, lung cancer, bladder cancer, endometrial cancer, bile duct cancer or breast cancer, comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean Cₘₐₓ of said Compound A of from about 13 ng/mL to about 3.8×10² ng/mL after administration to human subjects.

In further embodiment, the present invention provides an oral dosage form for treating stomach cancer, lung cancer, bladder cancer, endometrial cancer, bile duct cancer or breast cancer, comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean AUC₍₀₋ₜ₎ of said Compound A of from about 1.5×10² h*ng/mL to about 8.1×10³ h*ng/mL after administration to human subjects.

In further embodiment, the present invention provides an oral dosage form for treating stomach cancer, lung cancer, bladder cancer, endometrial cancer, bile duct cancer or breast cancer, comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean AUC₍₀₋ₜ₎ of said Compound A of from about 1.6×10² h*ng/mL to about 8.1×10³ h*ng/mL after administration to human subjects.

In further embodiment, the present invention provides an oral dosage form for treating stomach cancer, lung cancer, bladder cancer, endometrial cancer, bile duct cancer or breast cancer, comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean AUC₍₀₋ₜ₎ of said Compound A of from about 1.5×10² h*ng/mL to about 4.7×10³ h*ng/mL after administration to human subjects.

In further embodiment, the present invention provides an oral dosage form for treating stomach cancer, lung cancer, bladder cancer, endometrial cancer, bile duct cancer or breast cancer, comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean AUC₍₀₋ₜ₎ of said Compound A of from about 1.6×10² h*ng/mL to about 4.7×10³ h*ng/mL after administration to human subjects.

In further embodiment, the present invention provides an oral dosage form for treating stomach cancer, lung cancer, bladder cancer, endometrial cancer, bile duct cancer or breast cancer, comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean Cₘₐₓ of said Compound B of from about 12 ng/mL to about 1.6×10² ng/mL after administration to human subjects.

In further embodiment, the present invention provides an oral dosage form for treating stomach cancer, lung cancer, bladder cancer, endometrial cancer, bile duct cancer or breast cancer, comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean Cₘₐₓ of said Compound B of from about 12 ng/mL to about 1.1×10² ng/mL after administration to human subjects.

In further embodiment, the present invention provides an oral dosage form for treating stomach cancer, lung cancer, bladder cancer, endometrial cancer, bile duct cancer or breast cancer, comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean AUC₍₀₋ₜ₎ of said Compound B of from about 1.9×10² h*ng/mL to about 2.1×10³ h*ng/mL after administration to human subjects.

In further embodiment, the present invention provides an oral dosage form for treating stomach cancer, lung cancer, bladder cancer, endometrial cancer, bile duct cancer or breast cancer, comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean AUC₍₀₋ₜ₎ of said Compound B of from about 1.9×10² h*ng/mL to about 1.7×10³ h*ng/mL after administration to human subjects.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is single daily dose of about 30 mg to about 140 mg.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves Cₘₐₓ of said Compound A of from about 28 ng/mL to about 3.5×10² ng/mL after administration to the human subject.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves Cₘₐₓ of said Compound A of from about 28 ng/mL to about 2.3×10² ng/mL after administration to the human subject.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves AUC₍₀₋ₜ₎ of said Compound A of from about 2.2×10² h*ng/mL to about 7.2×10³ h*ng/mL after administration to the human subject.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves AUC₍₀₋ₜ₎ of said Compound A of from about 2.2×10² h*ng/mL to about 4.0×10³ h*ng/mL after administration to the human subject.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves AUC₍₀₋ₜ₎ of said Compound A of from about 2.2×10² h*ng/mL to about 3.9×10³ h*ng/mL after administration to the human subject.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves AUC_{(0-inf)} of said Compound A of from about 2.3×10² h*ng/mL to about 7.4×10³ h*ng/mL after administration to the human subject.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves AUC_{(0-inf)} of said Compound A of from about 2.3×10² h*ng/mL to about 4.1×10³ h*ng/mL after administration to the human subject.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves AUC_{(0-inf)} of said Compound A of from about 2.3×10² h*ng/mL to about 4.0×10³ h*ng/mL after administration to the human subject.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves Cₘₐₓ of said Compound B of from about 19 ng/mL to about 1.0×10² ng/mL after administration to the human subject.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves Cₘₐₓ of said Compound B of from about 19 ng/mL to about 64 ng/mL after administration to the human subject.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves AUC₍₀₋ₜ₎ of said Compound B of from about 2.7×10² h*ng/mL to about 1.6×10³ h*ng/mL after administration to the human subject.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves AUC₍₀₋ₜ₎ of said Compound B of from about 2.7×10² h*ng/mL to about 1.4×10³ h*ng/mL after administration to the human subject.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves AUC_{(0-inf)} of said Compound B of from about 2.9×10² h*ng/mL to about 1.7×10³ h*ng/mL after administration to the human subject.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said single daily dose achieves AUC_{(0-inf)} of said Compound B of from about 2.9×10² h*ng/mL to about 1.5×10³ h*ng/mL after administration to the human subject.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean Cₘₐₓ of said Compound A of from about 13 ng/mL to about 5.5×10² ng/mL after administration to human subjects.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean Cₘₐₓ of said Compound A of from about 13 ng/mL to about 3.8×10² ng/mL after administration to human subjects.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean AUC₍₀₋ₜ₎ of said Compound A of from about 1.5×10² h*ng/mL to about 8.1×10³ h*ng/mL after administration to human subjects.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean AUC₍₀₋ₜ₎ of said Compound A of from about 1.6×10² h*ng/mL to about 8.1×10³ h*ng/mL after administration to human subjects.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean AUC₍₀₋ₜ₎ of said Compound A of from about 1.5×10² h*ng/mL to about 4.7×10³ h*ng/mL after administration to human subjects.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean AUC₍₀₋ₜ₎ of said Compound A of from about 1.6×10² h*ng/mL to about 4.7×10³ h*ng/mL after administration to human subjects.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean Cₘₐₓ of said Compound B of from about 12 ng/mL to about 1.6×10² ng/mL after administration to human subjects.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean Cₘₐₓ of said Compound B of from about 12 ng/mL to about 1.1×10² ng/mL after administration to human subjects.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean AUC₍₀₋ₜ₎ of said Compound B of from about 1.9×10² h*ng/mL to about 2.1×10³ h*ng/mL after administration to human subjects.

In yet another embodiment, the present invention provides a method of treating stomach cancer, lung cancer, bladder cancer, endometrial cancer, bile duct cancer or breast cancer, comprising administering orally to a human subject in need thereof, an oral dosage form comprising a therapeutically effective amount of Compound A or a pharmaceutically acceptable salt thereof and at least one pharmaceutically acceptable excipient, wherein said therapeutically effective amount is repeated once-daily doses of about 30 mg to 140 mg, and said repeated once-daily doses achieve a mean AUC₍₀₋ₜ₎ of said Compound B of from about 1.9×10² h*ng/mL to about 1.7×10³ h*ng/mL after administration to human subjects.

In the present invention, the oral dosage form may comprise about 30 mg to about 140 mg, about 35 mg to about 140 mg, about 60 mg to about 140 mg, about 70 mg to about 140 mg, about 100 mg to about 140 mg, or 105 mg to about 140 mg of Compound A or a pharmaceutically acceptable salt thereof. In the present invention, the therapeutically effective amount of Compound A or a pharmaceutically acceptable salt thereof may be single daily dose of about 30 mg to about 140 mg, about 35 mg to about 140 mg, about 60 mg to about 140 mg, about 70 mg to about 140 mg, about 100 mg to about 140 mg, or 105 mg to about 140 mg. In the present invention, the therapeutically effective amount of Compound A or a pharmaceutically acceptable salt thereof may be repeated once-daily doses of about 30 mg to about 140 mg, about 35 mg to about 140 mg, about 60 mg to about 140 mg, about 70 mg to about 140 mg, about 100 mg to about 140 mg, or 105 mg to about 140 mg.

In the present invention, the oral dosage form at a single daily dose may achieve a mean Cₘₐₓ of said Compound A of from about 34 ng/mL to about 3.5×10² ng/mL, or from about 55 ng/mL to about 3.5×10² ng/mL after administration to human subjects.

In present invention, the oral dosage form at a single daily dose may achieve a mean Cₘₐₓ of said Compound A of from about 38 ng/mL to about 2.3×10² ng/mL, or from about 85 ng/mL to about 2.3×10² ng/mL after administration to human subjects.

In present invention, the oral dosage form at a single daily dose may achieve a mean AUC₍₀₋ₜ₎ of said Compound A of from about 2.7×10² h*ng/mL to about 7.2×10³ h*ng/mL, or from about 6.0×10² h*ng/mL to about 7.2×10³ h*ng/mL after administration to human subjects.

In present invention, the oral dosage form at a single daily dose may achieve a mean AUC₍₀₋ₜ₎ of said Compound A of from about 5.3×10² h*ng/mL to about 4.0×10³ h*ng/mL, or from about 1.0×10³ h*ng/mL to about 4.0×10³ h*ng/mL after administration to human subjects.

In present invention, the oral dosage form at a single daily dose may achieve a mean AUC₍₀₋ₜ₎ of said Compound A of from about 5.2×10² h*ng/mL to about 3.9×10³ h*ng/mL, or from about 1.0×10³ h*ng/mL to about 3.9×10³ h*ng/mL after administration to human subjects.

In present invention, the oral dosage form at a single daily dose may achieve a mean AUC_{(0-inf)} of said Compound A of from about 3.0×10² h*ng/mL to about 7.4×10³ h*ng/mL, or from about 6.4×10² h*ng/mL to about 7.4×10³ h*ng/mL after administration to human subjects.

In present invention, the oral dosage form at a single daily dose may achieve a mean AUC_{(0-inf)} of said Compound A of from about 3.1×10² h*ng/mL to about 7.4×10³ h*ng/mL, or from about 6.3×10² h*ng/mL to about 7.4×10³ h*ng/mL after administration to human subjects.

In present invention, the oral dosage form at a single daily dose may achieve a mean AUC_{(0-inf)} of said Compound A of from about 5.5×10² h*ng/mL to about 4.1×10³ h*ng/mL, or from about 1.1×10³ h*ng/mL to about 4.1×10³ h*ng/mL after administration to human subjects.

In present invention, the oral dosage form at a single daily dose may achieve a mean AUC_{(0-inf)} of said Compound A of from about 5.5×10² h*ng/mL to about 4.0×10³ h*ng/mL, or from about 1.1×10³ h*ng/mL to about 4.0×10³ h*ng/mL after administration to human subjects.

In present invention, the oral dosage form at a single daily dose may achieve a mean Cₘₐₓ of said Compound B of from about 16 ng/mL to about 1.0×10² ng/mL, or from about 34 ng/mL to about 1.0×10² ng/mL after administration to human subjects.

In present invention, the oral dosage form at a single daily dose may achieve a mean Cₘₐₓ of said Compound B of from about 35 ng/mL to about 64 ng/mL, or from about 38 ng/mL to about 64 ng/mL after administration to human subjects.

In present invention, the oral dosage form at a single daily dose may achieve a mean AUC₍₀₋ₜ₎ of said Compound B of from about 3.0×10² h*ng/mL to about 1.6×10³ h*ng/mL, or from about 4.9×10² h*ng/mL to about 1.6×10³ h*ng/mL after administration to human subjects.

In present invention, the oral dosage form at a single daily dose may achieve a mean AUC₍₀₋ₜ₎ of said Compound B of from about 6.5×10² h*ng/mL to about 1.4×10³ h*ng/mL, or from about 6.4×10² h*ng/mL to about 1.4×10³ h*ng/mL after administration to human subjects.

In present invention, the oral dosage form at a single daily dose may achieve a mean AUC_{(0-inf)} of said Compound B of from about 3.3×10² h*ng/mL to about 1.7×10³ h*ng/mL, or from about 5.3×10² h*ng/mL to about 1.7×10³ h*ng/mL after administration to human subjects.

In present invention, the oral dosage form at a single daily dose may achieve a mean AUC_{(0-inf)} of said Compound B of from about 6.8×10² h*ng/mL to about 1.5×10³ h*ng/mL, or from about 6.9×10² h*ng/mL to about 1.5×10³ h*ng/mL after administration to human subjects.

In present invention, the oral dosage form at repeated once-daily doses may achieve a mean Cₘₐₓ of said Compound A of from about 50 ng/mL to about 5.5×10² ng/mL, or from about 1.1×10² ng/mL to about 5.5×10² ng/mL after administration to human subjects.

In present invention, the oral dosage form at repeated once-daily doses may achieve a mean Cₘₐₓ of said Compound A of from about 50 ng/mL to about 3.8×10² ng/mL, or from about 1.1×10² ng/mL to about 3.8×10² ng/mL after administration to human subjects.

In present invention, the oral dosage form at repeated once-daily doses may achieve a mean AUC₍₀₋ₜ₎ of said Compound A of from about 4.9×10² h*ng/mL to about 8.1×10³ h*ng/mL, or from about 9.1×10² h*ng/mL to about 8.1×10³ h*ng/mL after administration to human subjects.

In present invention, the oral dosage form at repeated once-daily doses may achieve a mean AUC₍₀₋ₜ₎ of said Compound A of from about 5.0×10² h*ng/mL to about 8.1×10³ h*ng/mL, or from about 9.0×10² h*ng/mL to about 8.1×10³ h*ng/mL after administration to human subjects.

In present invention, the oral dosage form at repeated once-daily doses may achieve a mean AUC₍₀₋ₜ₎ of said Compound A of from about 4.9×10² h*ng/mL to about 4.7×10³ h*ng/mL, or from about 1.3×10³ h*ng/mL to about 4.7×10³ h*ng/mL after administration to human subjects.

In present invention, the oral dosage form at repeated once-daily doses may achieve a mean AUC₍₀₋ₜ₎ of said Compound A of from about 5.0×10² h*ng/mL to about 4.7×10³ h*ng/mL, or from about 1.3×10³ h*ng/mL to about 4.7×10³ h*ng/mL after administration to human subjects.

In present invention, the oral dosage form at repeated once-daily doses may achieve a mean Cₘₐₓ of said Compound B of from about 44 ng/mL to about 1.6×10² ng/mL, or from about 40 ng/mL to about 1.6×10² ng/mL after administration to human subjects.

In present invention, the oral dosage form at repeated once-daily doses may achieve a mean Cₘₐₓ of said Compound B of from about 44 ng/mL to about 1.1×10² ng/mL, or from about 55 ng/mL to about 1.1×10² ng/mL after administration to human subjects.

In present invention, the oral dosage form at repeated once-daily doses may achieve a mean AUC₍₀₋ₜ₎ of said Compound B of from about 5.6×10² h*ng/mL to about 2.1×10³ h*ng/mL, or from about 7.0×10² h*ng/mL to about 2.1×10³ h*ng/mL after administration to human subjects.

In present invention, the oral dosage form at repeated once-daily doses may achieve a mean AUC₍₀₋ₜ₎ of said Compound B of from about 5.6×10² h*ng/mL to about 1.7×10³ h*ng/mL, or from about 8.0×10² h*ng/mL to about 1.7×10³ h*ng/mL after administration to human subjects.

In present invention, Compound A can be prepared by a method known in the art, such as US 2014-235614 and WO/2016/152907.

Pharmaceutically acceptable salts may include, but are not limited to, inorganic acid salts; organic carboxylates; organic sulfonates; amino acid salts; quaternary amine salts; alkaline metal salts; and alkaline-earth metal salts. Preferred pharmaceutically acceptable salts include succinate such as 1.5 succinate.

Oral dosage forms of the present invention include capsules, granules, lozenges, pellets, pills, powders, suspensions, tablets, preferably capsules, granules, pellets, pills, tablets.

The oral dosage form of the present invention may be prepared, using standard techniques and manufacturing processes generally known in the art. See, e.g. the monograph of Japanese Pharmacopoeia 16 edition or General Chapter <1151> Pharmaceutical Dosage Forms of U.S. Pharmacopoeia-NF (37).

### EXAMPLES

The following examples illustrate various aspects of the present invention. They are not to be construed to limit the claims in any manner whatsoever.

Compound A 1.5 succinate was synthesized according to the method described in WO/2016/152907.

The following study was carried out in order to evaluate tolerability and safety when Compound A 1.5 succinate was orally administered to patients with solid tumor. We attempted to determine the maximum tolerated dose (MTD) by evaluating the dose limiting toxicity (DLT) when Compound A 1.5 succinate was orally administered to patients with solid tumor. Each dose in Examples is represented as a dose of Compound A in free form.

### Methods

A Modified Toxicity Probability Interval (mTPI) design was employed to determine the MTD of Compound A. Each subject was assigned a dose of Compound A in accordance with the rules of the mTPI design based on a target dose-limiting toxicity (DLT) rate of 25% and the corresponding three toxicity probability intervals that was defined as 20 to 30% (proper dosing), 0 to 20% (underdosing) and 30 to 100% (overdosing). The entire dose assignment decision rule can be precalculated under the mTPI design and presented in two-way table as below.

TABLE 1
Decision Rule for Dose Assignment

| | | Number of Subjects Treated at the Current Dose | | | | |
|---|---|---|---|---|---|---|
| | | 2 | 4 | 6 | 8 | 10 |
| Number | 0 | E | E | E | E | E |
| of | 1 | D^{a} | S | S | E | E |
| Subjects | 2 | D, U | D | S | S^{b} | S |
| with | 3 | | D, U | D | S | S^{b} |
| DLT | 4 | | D, U | D, U | D, U | S |
| | 5 | | | D, U | D, U | D, U |

E = Escalate to the next higher dose,
S = Stay at the current dose,
D = De-escalate to the next lower dose,
U = current dose is unacceptably toxic (i.e., Do not re-enter the current dose)
Target DLT rate at MTR = 25% and its equivalence toxicity interval = 20 to 30%. Cohort size = 2 subjects
Display for more than 10 subjects at the current dose is omitted
^{a}Extra subjects may be added for the starting dose.
^{b}Early closure of registration may be considered.

### Administration Schedule

1) Cycle 0 (for 7 days)

In order to evaluate the PK when administered as a single dose, a single dose of Compound A for each treatment group (at the corresponding dosage for that group) was administered on Day 1. Compound A was administered when fasted, immediately after waking up with at least 10 hours fasting. Taking any meal was prohibited for 2 hours after administration and only drinking water was allowed.

2) Cycle 1 or later (28 day cycles)

Cycle 1 was started between 8 and 10 days after dosing in Cycle 0 and Compound A was administered continuously once daily. Compound A was administered at least 2 hours after breakfast, and any food intake was prohibited for 1 hour after administration. However, on Day 8 of Cycle 1, Compound A was administered immediately after waking up while the subject was fasted after at least 10 hours of overnight fasting in order to evaluate the PK. Taking any meals was prohibited for 2 hours after administration and only drinking water was allowed.

### Setting of Starting Dose

The starting dose of Compound A in this study was set based on the guidelines in “Nonclinical Evaluation for Anticancer Pharmaceuticals” (ICH S9; PFSB/ELD Notification No. 0604-1, dated Jun. 4, 2010). According to this guideline, a common approach for many small-molecules is to set a starting dose at 1/10 the Severely Toxic Dose in 10% of the animals (STD 10; dose that is associated with lethality, life-threatening toxicities, or irreversible toxicities) in rodents, or at ⅙ the Highest Non-Severely Toxic Dose (HNSTD) in the case where non-rodents are the most appropriate test species. Considering subject safety, the 1.46 mg dose that was calculated from toxicity studies in rats (which are highly sensitivity to toxicity) was thus adopted and the starting dose in this study was set as 1 mg, a dose below the 1.46 mg dose.

### Inclusion Criteria

(1) Subjects age>=20 years at the time of informed consent
(2) Subjects with a histological and/or cytological diagnosis of solid tumor
(3) Subjects who failed standard therapies, or for which no appropriate treatment is available.
(4) Corrected serum calcium<=ULN
(5) Serum phosphate<=ULN
(6) Subjects with Performance Status (PS) score of 0-1 established by Eastern Cooperative Oncology Group (ECOG)

### Exclusion Criteria

(1) Subjects with brain metastasis that is associated with clinical symptoms or require treatment
(2) Medical history of clinically significant cardiovascular impairment
(3) Current evidence or history of corneal disorder>=Grade 2

### DLT Criteria

(1) Grade 4 neutropenia that persists for more than 7 days or febrile neutropenia
(2) Grade 4 thrombocytopenia or Grade 3 thrombocytopenia that requires blood transfusion
(3) Any Grade 3 or higher non-hematological toxicity with the exception of:
   a) Abnormal clinical laboratory values with no clinical significance.
   b) Any events which can be managed and controlled to Grade 2 or less by maximal medical management.
(4) New calcification that is considered clinically significance in such as soft tissue, kidney, intestine, heart or lung confirmed by images
(5) Hyperphosphatemia defined as follows
   a) >7 mg/dL>7d despite phosphate lowering therapies
   b) >9 mg/dL despite phosphate lowering therapies.
(6) Development of any toxicity that is considered to be related to Compound A and where treatment interruption for 8 days or more from Cycle 0 to Cycle 1 is necessary.

### Results

### Patient Characteristics

TABLE 2

| | | No. of | |
|---|---|---|---|
| | | Subjects | |
| | | (%) | |
| | | (N = 24) | |
| Age, Years | Median | 65 | |
| | Range | 42-75 | |
| Gender | Male | 11 | (46) |
| | Female | 13 | (54) |
| ECOG-PS | 0 | 17 | (71) |
| | 1 | 7 | (29) |
| No. of prior | ≤2 | 9 | (38) |
| chemotherapy | ≥3 | 15 | (63) |
| Cancer type | Cholangiocarcinoma | 8 | (33) |
| | Pancreatic cancer | 3 | (13) |
| | Endometrial cancer | 2 | (8) |
| | Cancer of unknown primary | 2 | (8) |
| | Other | 9 | (38) |

### Summary of Dose Escalation Study

Among the patients treated with once-daily dosing of Compound A: 1 mg (2 patients), 2 mg (2 patients), 4 mg (2 patients), 8 mg (2 patients), 16 mg (2 patients), 30 mg (2 patients), 60 mg (3 patients), 100 mg (3 patients), 140 mg (3 patients) and 180 mg (3 patients), one patient at 180 mg dosing experienced the DLT (Grade 3 AST/ALT increased). The MTD was not defined and the recommended dose was determined to be 140 mg once daily.

Treatment emergent adverse events (>=5%) are as shown in the following table.

TABLE 3

| | 1-30 mg | | 60 mg | | 100 mg | | 140 mg | | 180 mg | | Total | |
|---|---|---|---|---|---|---|---|---|---|---|---|---|
| | (N = 12) | | (N = 3) | | (N = 3) | | (N = 3) | | (N = 3) | | (N = 24) | |
| | Any | | Any | | Any | | Any | | Any | | Any | |
| Toxicities | Gr | ≥Gr3 | Gr | ≥Gr3 | Gr | ≥Gr3 | Gr | ≥Gr3 | Gr | ≥Gr3 | Gr | ≥Gr3 |
| Hyperphosphatemia | — | — | — | — | 3 (100) | — | 3 (100) | — | 3 (100) | — | 9 (38) | — |
| Creatinine | 2 (17) | — | 2 (67) | — | 1 (33) | — | 3 (100) | — | 1 (33) | — | 9 (38) | — |
| increased | | | | | | | | | | | | |
| ATL | — | — | 1 (33) | — | — | — | 3 (100) | — | 2 (67) | 2 (67) | 6 (25) | 2 (8) |
| increased | | | | | | | | | | | | |
| Diarrhoea | 1 (8) | — | 2 (67) | — | — | — | 1 (33) | — | 3 (100) | — | 6 (25) | — |
| Lipase | 1 (8) | — | 1 (33) | — | 1 (33) | — | 1 (33) | — | 1 (33) | — | 5 (21) | — |
| increased | | | | | | | | | | | | |
| Nausea | — | — | 1 (33) | — | 1 (33) | — | 1 (33) | — | 1 (33) | — | 5 (21) | — |
| AST | — | — | — | — | — | — | 2 (67) | — | 2 (67) | 1 (33) | 4 (17) | — |
| increased | | | | | | | | | | | | |
| PPE | — | — | 1 (33) | — | — | — | 1 (33) | — | 2 (67) | — | 4 (17) | — |
| syndrome | | | | | | | | | | | | |
| ALP | — | — | — | — | 1 (33) | — | 2 (67) | — | — | — | 3 (13) | — |
| increased | | | | | | | | | | | | |
| Anaemia | — | — | 1 (33) | — | 1 (33) | — | 1 (33) | — | — | — | 3 (13) | — |
| Dysgeusia | — | — | — | — | — | — | 2 (67) | — | 1 (33) | — | 3 (13) | — |
| Retinal | — | — | — | — | 2 (67) | — | 1 (33) | — | — | — | 3 (13) | — |
| detachment | | | | | | | | | | | | |
| Vomiting | — | — | 1 (33) | — | — | — | 1 (33) | — | 1 (33) | 1 (33) | 3 (13) | 1 (4) |
| Constipation | 1 (8) | — | — | — | — | — | 1 (33) | — | — | — | 2 (8) | — |
| Decreased | — | — | — | — | — | — | 1 (33) | — | 1 (33) | 1 (33) | 2 (8) | 1 (4) |
| appetite | | | | | | | | | | | | |
| Lymphocyte | | | | | | | | | | | | |
| count | — | — | 1 (33) | 1 (33) | — | — | — | — | 1 (33) | — | 2 (8) | — |
| decreased | | | | | | | | | | | | |
| Malaise | — | — | 1 (33) | — | — | — | 1 (33) | — | — | — | 2 (8) | — |
| Neutrophil | | | | | | | | | | | | |
| count | — | — | — | — | — | — | — | — | 2 (67) | 1 (33) | 2 (8) | 1 (4) |
| decreased | | | | | | | | | | | | |
| Onychomadesis | — | — | — | — | — | — | 1 (33) | — | 1 (33) | — | 2 (8) | — |
| Stomatitis | — | — | — | — | 1 (33) | — | — | — | 1 (33) | — | 2 (8) | — |
| White | — | — | 1 (33) | — | — | — | — | — | 1 (33) | — | 2 (8) | — |
| blood cell | | | | | | | | | | | | |
| count | | | | | | | | | | | | |
| decreased | | | | | | | | | | | | |

Serious adverse events were reported in 3 patients (dyspnoea in a patient in 8 mg cohort, tumor pain aggravation in a patient in 8 mg cohort and pyrexia in a patient at 30 mg cohort). However, none of them were considered to be related to Compound A.

No death or adverse events leading to study drug withdrawal were reported.

The adverse events leading to dose reductions were ALT increased (2 patients), palmar-plantar erythrodysesthesia syndrome (2 patients) and AST increased (1 patient).

The adverse events leading to dose interruptions were nausea (3 patients), vomiting (2 patients), anorexia (2 patients), pyrexia (2 patients), common cold (1 patient), neutrophil count decreased (1 patient), macular edema (1 patient) and palmar-plantar erythrodysesthesia syndrome (1 patient).

### Pharmacokinetics

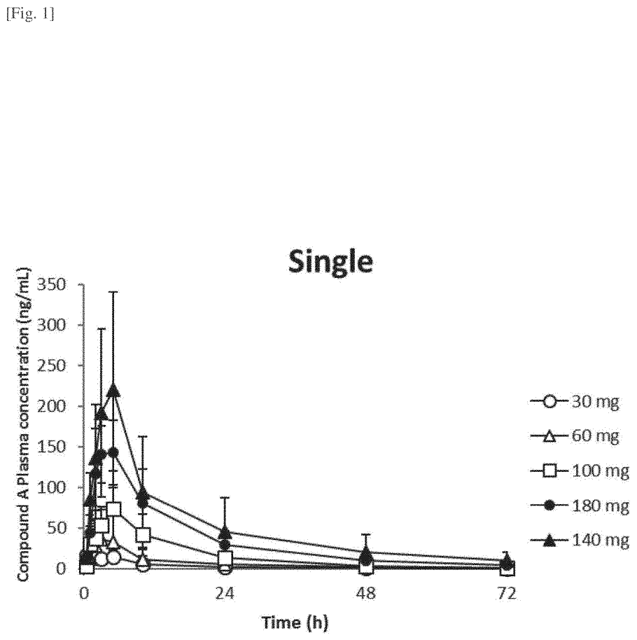

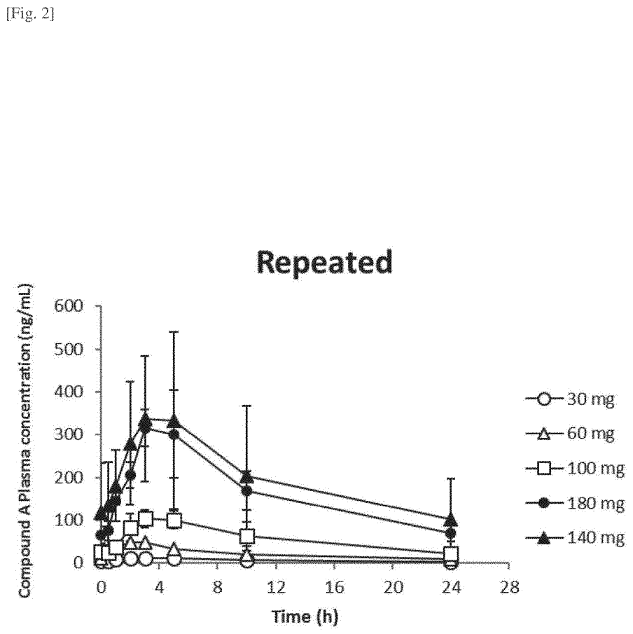

Plasma concentration profile of Compound A following a single dose and repeated doses of Compound A 1.5 succinate are shown in FIG. 1 and FIG. 2, respectively. The profile shown in FIG. 2 is that at steady state.

The pharmacokinetic parameters of Compound A following the single dose and repeated doses of Compound A 1.5 succinate are shown in the following table. The pharmacokinetic parameters of Compound A following repeated doses of Compound A 1.5 succinate shown below are those at steady state.

TABLE 4

| | | | | |
|---|---|---|---|---|
| 30 mg | 60 mg | 100 mg | 140 mg | 180 mg |
| (n = 2) | (n = 3) | (n = 3) | (n = 3) | (n = 3) |

Single

| | | | | | | |
|---|---|---|---|---|---|---|
| t_{1/2} | (h) | 22.8 | 23.1 ± 7.65 | 15.1 ± 1.77 | 26.4 ± 12.2 | 23.6 ± 10.2 |
| tₘₐₓ | (h) | 3 (1-5) | 3 (3-5) | 5 (3-5) | 5 (3-5) | 2 (2-5) |
| Cₘₐₓ | (ng/mL) | 28.1 | 38.7 ± 4.69 | 85.9 ± 30.8 | 227 ± 118.0 | 154 ± 38.4 |
| AUC₍₀₋ₜ₎ | (h · ng/mL) | 220 | 529 ± 250 | 1090 ± 483 | 3900 ± 3230 | 2540 ± 924 |
| AUC_{(0-inf)} | (h · ng/mL) | 236 | 551 ± 240 | 1120 ± 488 | 4000 ± 3340 | 2570 ± 907 |
| Vz/F | (L) | 4230 | 3820 ± 1280 | 2310 ± 1370 | 1700 ± 650 | 2560 ± 1490 |
| CL/F | (L/h) | 128 | 122 ± 42.5 | 103 ± 48.9 | 60.4 ± 51.6 | 75.0 ± 22.0 |

Repeat

| | | | | | | |
|---|---|---|---|---|---|---|
| tₘₐₓ | (h) | 4 (3-5) | 2.5 (2-3)^{a} | 3 (2-5) | 5 (3-5) | 5 (3-5) |
| Cₘₐₓ | (ng/mL) | 13.3 | 50.5^{a} | 116 ± 3.79 | 372 ± 173 | 337 ± 60.3 |
| AUC₍₀₋ₜ₎ | (h · ng/mL) | 158 | 499^{a} | 1330 ± 417 | 4700 ± 3380 | 3850 ± 777 |
| Cₘᵢₙ | (ng/mL) | 2.48 | 8.28^{a} | 18.7 ± 7.95 | 101 ± 94.5 | 61.4 ± 19.9 |
| C_{avg} | (ng/mL) | 6.58 | 20.8^{a} | 55.2 ± 17.3 | 196 ± 141 | 161 ± 32.3 |

^{a}n = 2
Cmax and AUC increased with increasing dose with tmax of 2-5 hr and mean t½ of 15.1-26.4 hr.

The pharmacokinetic parameters of Compound A following the single dose and repeated doses of Compound A 1.5 succinate updated after obtaining the above data are shown in the following table. The pharmacokinetic parameters of Compound A following repeated doses of Compound A 1.5 succinate shown below are those at steady state.

TABLE 5

| | | | | |
|---|---|---|---|---|
| 30 mg | 60 mg | 100 mg | 140 mg | 180 mg |
| (n = 2) | (n = 3) | (n = 3) | (n = 3) | (n = 3) |

Single

| | | | | | | |
|---|---|---|---|---|---|---|
| t_{1/2} | (h) | 22.7 | 23.1 ± 7.63 | 15.2 ± 1.84 | 26.4 ± 12.4 | 23.7 ± 10.4 |
| tₘₐₓ | (h) | 3.04 (0.93-5.15) | 3.00 (2.97-4.98) | 4.95 (2.97-5.10) | 4.88 (3.00-5.08) | 2.12 (1.95-5.00) |
| Cₘₐₓ | (ng/mL) | 28.1 | 38.7 ± 4.69 | 85.9 ± 30.8 | 227 ± 118 | 154 ± 38.4 |
| AUC₍₀₋ₜ₎ | (h · ng/mL) | 222 | 533 ± 257 | 1080 ± 474 | 3960 ± 3230 | 2570 ± 933 |
| AUC_{(0-inf)} | (h · ng/mL) | 239 | 554 ± 247 | 1120 ± 479 | 4050 ± 3340 | 2610 ± 921 |
| Vz/F | (L) | 4180 | 3790 ± 1240 | 2310 ± 1360 | 1670 ± 617 | 2550 ± 1500 |
| CL/F | (L/h) | 127 | 121 ± 43.1 | 103 ± 48.2 | 59.0 ± 50.2 | 74.2 ± 21.8 |

Repeat

| | | | | | | |
|---|---|---|---|---|---|---|
| tₘₐₓ | (h) | 3.99 (3.00-4.97) | 2.54 (2.07-3.00) ^{a} | 2.97 (2.03-5.00) | 5.00 (2.98-5.08) | 5.00 (3.05-5.08) |
| Cₘₐₓ | (ng/mL) | 13.3 | 50.5^{a} | 116 ± 3.79 | 372 ± 173 | 337 ± 60.3 |
| AUC₍₀₋ₜ₎ | (h · ng/mL) | 162 | 507^{a} | 1340 ± 437 | 4700 ± 3380 | 3860 ± 764 |
| Cₘᵢₙ | (ng/mL) | 4.50 | 11.0^{a} | 22.3 ± 8.48 | 117 ± 116 | 64.8 ± 19.4 |
| C_{avg} | (ng/mL) | 6.75 | 21.1^{a} | 55.91 ± 8.2 | 196 ± 141 | 161 ± 32.3 |

Data is shown as mean ± SD except for tmax; for tmax, median (minimum − maximum) is shown.
PK prameters were calculated by NCA method using prompt results for plasma concentrations, nominal time.
Cmax and AUC increased with increasing dose with median tmax of 2-5 hrs and mean t½ of 15.2-26.6 hrs.
^{a}n = 2

The pharmacokinetic parameters of Compound B following the single dose and repeated doses of Compound A 1.5 succinate are shown in the following table. The pharmacokinetic parameters of Compound B following repeated doses of Compound A 1.5 succinate shown below are those at steady state.

TABLE 6

| | | | | |
|---|---|---|---|---|
| 30 mg | 60 mg | 100 mg | 140 mg | 180 mg |
| (n = 2) | (n = 3) | (n = 3) | (n = 3) | (n = 3) |

Single

| | | | | | | |
|---|---|---|---|---|---|---|
| t_{1/2} | (h) | 21.5 | 26.1 ± 10.2 | 20.0 ± 0.794 | 40.50 ± 6.46 | 39.9 ± 6.27 |
| tₘₐₓ | (h) | 3.59 (2.02-5.15) | 4.98 (3.00-5.07) | 4.95 (2.97-5.10) | 5.00 (4.88-5.08) | 5.00 (3.00-5.02) |
| Cₘₐₓ | (ng/mL) | 19.2 | 35.1 ± 19.1 | 38.4 ± 4.36 | 63.8 ± 36.1 | 50.7 ± 20.7 |
| AUC₍₀₋ₜ₎ | (h* ng/mL) | 274 | 652 ± 352 | 647 ± 153 | 1350 ± 233 | 1320 ± 693 |
| AUC_{(0-inf)} | (h* ng/mL) | 297 | 687 ± 354 | 699 ± 163 | 1410 ± 216 | 1370 ± 703 |

repeated

| | | | | | | |
|---|---|---|---|---|---|---|
| tₘₐₓ | (h) | 3.49 (1.97-5.00) | 2.54 (2.07-3.00) ^{a} | 5.00 (4.98-5.05) | 5.00 (2.98-5.08) | 5.00 (3.05-5.08) |
| Cₘₐₓ | (ng/mL) | 12.4 | 44.2 ^{a} | 55.4 ± 14.5 | 106 ± 51.8 | 98.0 ± 39.5 |
| AUC₍₀₋ₜ₎ | (h* ng/mL) | 194 | 565 ^{a} | 805 ± 195 | 1620 ± 460 | 1500 ± 686 |
| Cₘᵢₙ | (ng/mL) | 5.19 | 12.3 ^{a} | 17.4 ± 7.37 | 41.5 ± 10.9 | 32.8 ± 13.0 |
| C_{avg} | (ng/mL) | 7.71 | 22.6 ^{a} | 32.3 ± 7.79 | 66.1 ± 18.8 | 60.4 ± 27.7 |

Data is shown as mean ± SD except tₘₐₓ; for tₘₐₓ median (minimum − maximum) is shown.
^{a} n = 2

### Anti-Tumor Activity

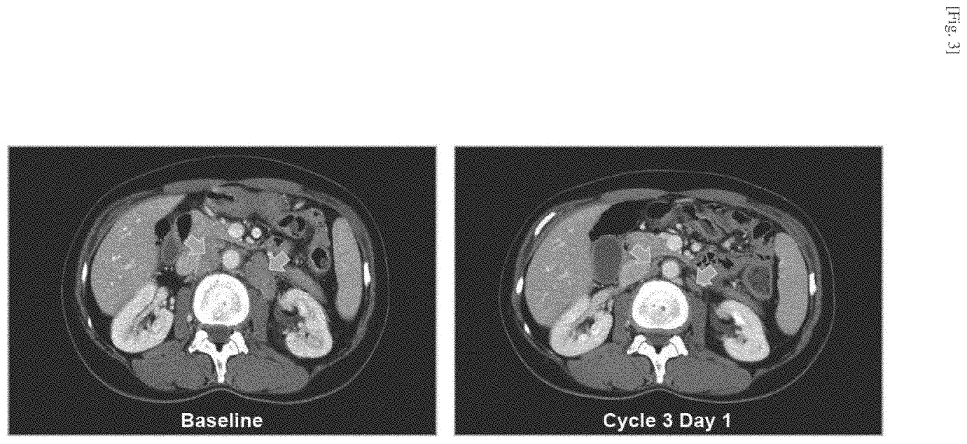

Compound A was administered once daily at 180 mg to a patient (45 years-old woman) with FGFR2 gene amplified diffused type gastric cancer (poorly differentiated adenocarcinoma). CT images of the patient are shown in FIG. 3. The left image is that before the administration and the right image is that on Day 1 of Cycle 3. The tumor size reduced significantly by the administration of Compound A.

When Compound A was administered once daily at 30 mg to a patient with FGFR2-fusion gene positive (78%) intrahepatic cholangiocarcinoma, the tumor size reduced by about 9%.

### Pharmacodynamics (PD)

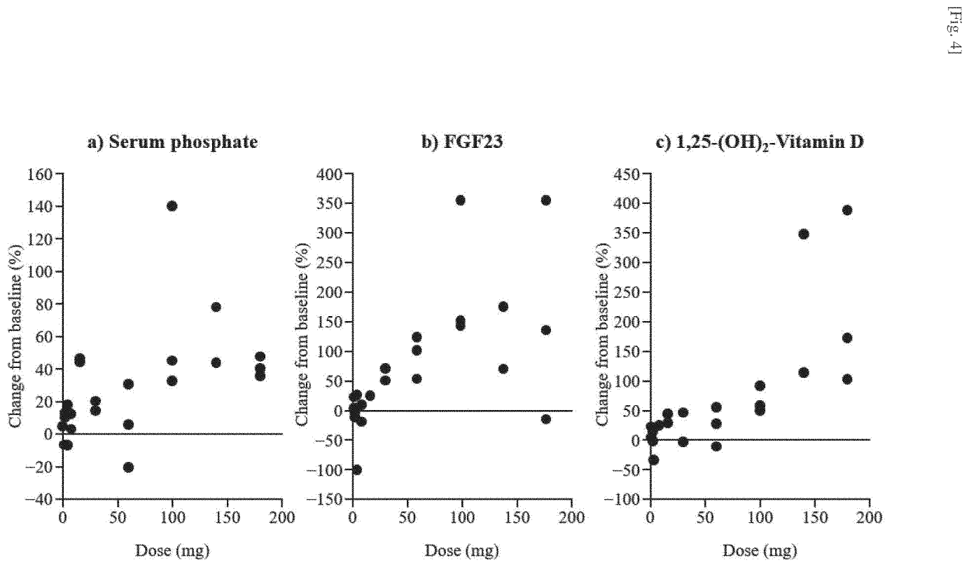

Serum concentrations of Phosphate, FGF23 and 1,25-(OH)₂-Vitamin D, PD markers of FGFR pathway inhibition were measured before the administration of Compound A and on Day 15 of Cycle 1. Changes in concentration during administration of Compound A were shown in FIG. 4. The administration of Compound A induced dose-dependent increases in each marker, and these increases reached maximum at approximately 100-140 mg once-daily dosing.

1. An oral dosage form comprising about 30 mg to about 140 mg of Compound A or a pharmaceutically acceptable salt thereof and at least one pharmaceutically acceptable excipient, wherein said Compound A is 5-((2-(4-(1-(2-hydroxyethyl)piperidin-4-yl)benzamide)pyridine-4-yl)oxy)-6-(2-methoxyethoxy)-N-methyl-1H-indole-1-carboxamide represented by Formula (I):

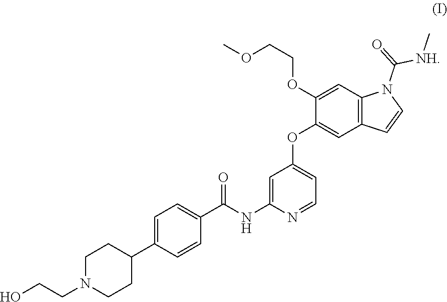 [SMILES: CNC(=O)n1ccc2cc(Oc3ccnc(NC(=O)c4ccc(C5CCN(CCO)CC5)cc4)c3)c(OCCOC)cc21]

2. The oral dosage form of claim 1, wherein said oral dosage form at a single daily dose achieves a mean Cₘₐₓ of said Compound A of from about 28 ng/mL to about 3.5×10² ng/mL after administration to human subjects.

3. The oral dosage form of claim 1, wherein said oral dosage form at a single daily dose achieves a mean AUC₍₀₋ₜ₎ of said Compound A of from about 2.2×10² h*ng/mL to about 7.2×10³ h*ng/mL after administration to human subjects.

4. The oral dosage form of claim 1, wherein said oral dosage form at a single daily dose achieves a mean AUC_{(0-inf)} of said Compound A of from about 2.3×10² h*ng/mL to about 7.4×10³ h*ng/mL after administration to human subjects.

5. The oral dosage form of claim 1, wherein said oral dosage form at a single daily dose achieves a mean Cₘₐₓ of Compound B of from about 19 ng/mL to about 1.0×10² ng/mL after administration to human subjects, and said Compound B is 6-(2-Methoxyethoxy)-N-methyl-5-((2-(4-(piperidin-4-yl)benzamide)pyridin-4-yl)oxy)-1H-indole-1-carboxamide represented by Formula (II):

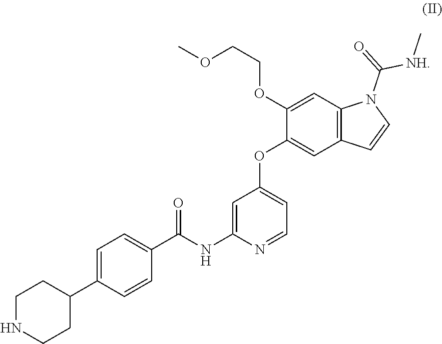 [SMILES: CNC(=O)n1ccc2cc(Oc3ccnc(NC(=O)c4ccc(C5CCNCC5)cc4)c3)c(OCCOC)cc21]

6. The oral dosage form of claim 1, wherein said oral dosage form at a single daily dose achieves a mean AUC₍₀₋ₜ₎ of Compound B of from about 2.7×10² h*ng/mL to about 1.6×10³ h*ng/mL after administration to human subjects, and said Compound B is 6-(2-Methoxyethoxy)-N-methyl-5-((2-(4-(piperidin-4-yl)benzamide)pyridin-4-yl)oxy)-1H-indole-1-carboxamide represented by Formula (II):

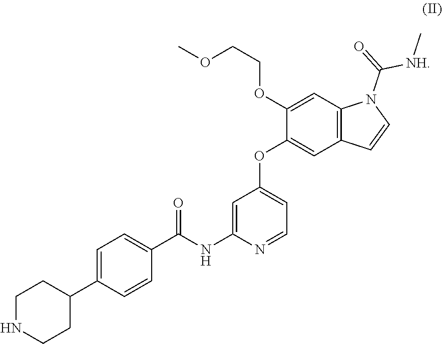 [SMILES: CNC(=O)n1ccc2cc(Oc3ccnc(NC(=O)c4ccc(C5CCNCC5)cc4)c3)c(OCCOC)cc21]

7. The oral dosage form of claim 1, wherein said oral dosage form at a single daily dose achieves a mean AUC_{(0-inf)} of Compound B of from about 2.9×10² h*ng/mL to about 1.7×10³ h*ng/mL after administration to human subjects, and said Compound B is 6-(2-Methoxyethoxy)-N-methyl-5-((2-(4-(piperidin-4-yl)benzamide)pyridin-4-yl)oxy)-1H-indole-1-carboxamide represented by Formula (II):

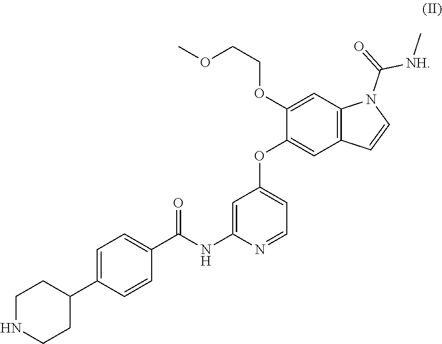 [SMILES: CNC(=O)n1ccc2cc(Oc3ccnc(NC(=O)c4ccc(C5CCNCC5)cc4)c3)c(OCCOC)cc21]

8. An oral dosage form comprising a therapeutically effective amount of Compound A or a pharmaceutically acceptable salt thereof and at least one pharmaceutically acceptable excipient, wherein said therapeutically effective amount is single daily dose of about 30 mg to about 140 mg, and said Compound A is 5-((2-(4-(1-(2-hydroxyethyl)piperidin-4-yl)benzamide)pyridine-4-yl)oxy)-6-(2-methoxyethoxy)-N-methyl-1H-indole-1indole-1-carboxamide represented by Formula (I):

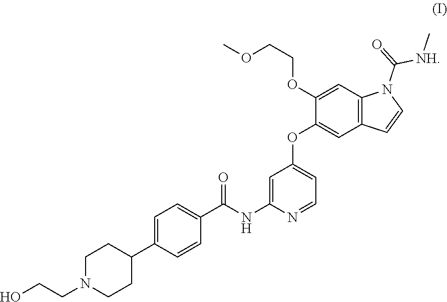 [SMILES: CNC(=O)n1ccc2cc(Oc3ccnc(NC(=O)c4ccc(C5CCN(CCO)CC5)cc4)c3)c(OCCOC)cc21]

9. The oral dosage form of claim 8, wherein said single daily dose achieves a mean Cₘₐₓ of said Compound A of from about 28 ng/mL to about 3.5×10² ng/mL after administration to human subjects.

10. The oral dosage form of claim 8, wherein said single daily dose achieves a mean AUC₍₀₋ₜ₎ of said Compound A of from about 2.2×10² h*ng/mL to about 7.2×10³ h*ng/mL after administration to human subjects.

11. The oral dosage form of claim 8, wherein said single daily dose achieves a mean AUC_{(0-inf)} of said Compound A of from about 2.3×10² h*ng/mL to about 7.4×10³ h*ng/mL after administration to human subjects.

12. The oral dosage form of claim 8, wherein said single daily dose achieves a mean Cₘₐₓ of Compound B of from about 19 ng/mL to about 1.0×10² ng/mL after administration to human subjects, and said Compound B is 6-(2-Methoxyethoxy)-N-methyl-5-((2-(4-(piperidin-4-yl)benzamide)pyridin-4-yl)oxy)-1H-indole-1-carboxamide represented by Formula (II):

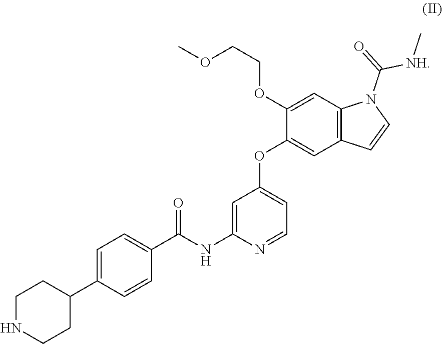 [SMILES: CNC(=O)n1ccc2cc(Oc3ccnc(NC(=O)c4ccc(C5CCNCC5)cc4)c3)c(OCCOC)cc21]

13. The oral dosage form of claim 8, wherein said single daily dose achieves a mean AUC₍₀₋ₜ₎ of Compound B of from about 2.7×10² h*ng/mL to about 1.6×10³ h*ng/mL after administration to human subjects, and said Compound B is 6-(2-Methoxyethoxy)-N-methyl-5-((2-(4-(piperidin-4-yl)benzamide)pyridin-4-yl)oxy)-1H-indole-1-carboxamide represented by Formula (II):

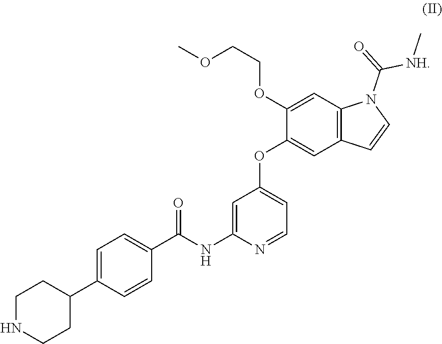 [SMILES: CNC(=O)n1ccc2cc(Oc3ccnc(NC(=O)c4ccc(C5CCNCC5)cc4)c3)c(OCCOC)cc21]

14. The oral dosage form of claim 8, wherein said single daily dose achieves a mean AUC_{(0-inf)} of Compound B of from about 2.9×10² h*ng/mL to about 1.7×10³ h*ng/mL after administration to human subjects, and said Compound B is 6-(2-Methoxyethoxy)-N-methyl-5-((2-(4-(piperidin-4-yl)benzamide)pyridin-4-yl)oxy)-1H-indole-1-carboxamide represented by Formula (II):

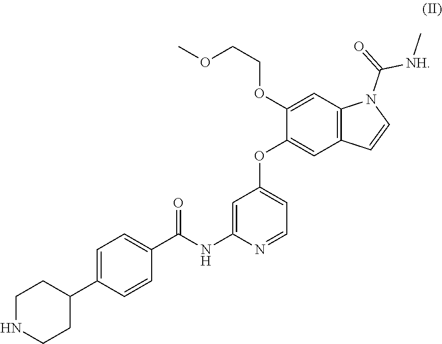 [SMILES: CNC(=O)n1ccc2cc(Oc3ccnc(NC(=O)c4ccc(C5CCNCC5)cc4)c3)c(OCCOC)cc21]

15. A method of treating stomach cancer, lung cancer, bladder cancer, endometrial cancer, bile duct cancer or breast cancer in a human subject in need thereof, comprising administering the oral dosage form of claim 1 to the human subject.
